(19) United States
(12) Patent Application Publication
(10) Pub. No.: US 2013/0079694 A1
(43) Pub. Date: Mar. 28, 2013

(54) Device Used to Realign Hammertoe Deformities

(71) Applicant: John Michael Aquino, Amherst, NY (US)

(72) Inventor: John Michael Aquino, Amherst, NY (US)

(21) Appl. No.: 13/417,911

(22) Filed: Mar. 12, 2012

Related U.S. Application Data
(60) Provisional application No. 61/537,937, filed on Sep. 22, 2011.

(51) Int. Cl. A61F 5/00

(52) U.S. Cl. 602/30

(57) ABSTRACT

A device for the realignment of hammer toe deformities comprising a dorsal piece and a plantar piece parallel thereto. The dorsal piece and the plantar piece are held together by a first and a second connecting arm. The device being of a resilient material and of a unitary construction is suitably sized and shaped to fit around the wearer's foot, within the wearer's shoe in a position wherein the dorsal piece rests across the bases of at least two of the central three toes of the wearer's foot and the plantar piece is tapered laterally and is comma shaped to fit in the sulci formed by the central three toes of the wearer's foot. The first connecting arm extends through the interspace between the hallux and the second toe and the second connecting arm extends through the interspace between the third toe and the fourth toe.

This application claims priority of U.S. Provisional Patent Application of John Michael Aquino, Ser. No. 61/537,937 for A DEVICE USED TO REALIGN HAMMERTOE DEFORMITIES, filed on Sep. 22, 2011.

### BACKGROUND OF THE INVENTION

This patent application pertains to a device used to realign hammertoe deformities. More specifically, this patent application pertains to a device used to realign hammertoe deformities by provision of a device designed to be worn between the central toes of the wearer.

A device to alleviate and/or realign hammertoe deformities, and/or treat hallux valgus, has been the focus of numerous patents. For instance, U.S. patent application 2010/0249687 to Goswami et al. presents a number of braces for treatment of toe ailments. The braces are used to support the metatarsophalangeal joint, reducing or maintaining the intermetatarsal angle, and/or enhancing or maintaining alignment of the hallux, or big toe. The braces are intended to treat individuals suffering from hallux valgus. However, the braces suffer significant drawbacks. First, the braces wrap the entire midfoot of the wearer leading to restricted movement and user discomfort. This discomfort will cause sufferers to stop using the device. Further, the braces are limited to the treatment of hallux vulgas and cannot be used to treat hammertoe.

In U.S. Pat. No. 7,396,338 to Huber et al. provides an orthopedic device for correction of hallux valgus. The device consists of a support which wraps the midfoot of the wearer with the support having a fastening portion which encircles the hallux. A flexible splint extends from the fastening portion to the support along the inner side of the foot. The splint applies force to the outer edge of the hallux thereby assisting in realigning the hallux in alleviation of hallux valgus. While purporting to aid in relieving hallux valgus, the device is limited as it cannot treat hammertoe deformities.

U.S. Pat. No. 6,318,373 to Kasahara discloses a corrective support to correct for hallux valgus and digitus varus minimi thereby allowing for the maintenance of outer and inner arches of the plantar sole. The device is constructed of a sleeve which wraps around the midfoot of the wearer. A pushing band pushes against the inward or outward prominent metatarsophalangeal. A pair of reinforcements protrudes outwardly from the sleeve with one reinforcement located between the first and second toes with the second reinforcement located between the fourth and fifth toes. The reinforcements apply an outward push force to assist in maintaining the vertical and lateral arches of the wearer. Although providing a pushing force to the first and fifth toe, the device presented by Kashara does not alleviate hammertoe deformities.

Therefore, it can be seen that there is a need for a device that can be worn in the shoe that splints the toes and eliminates pressure on the tops and tips of the toes. There is a need for a device that provides relief of lesser hammertoe deformities and associated corns on the tops of the tips of the toes. In alternative embodiments, the device further incorporates a buttress modification to help straighten hallux valgus or bunion deformities.

### SUMMARY OF THE INVENTION

It is the object of the invention to provide a toe realignment and toe protecting device which includes a dorsal and plantar piece and connecting arms between the toes.

It is another object of the invention to provide a one-piece construction toe realignment and toe protecting device which includes a dorsal and plantar piece and connecting arms between the toes, the device being made of a resilient material, preferably silicone, viscoelastic gel or moldable rubber.

Still another object of the invention is to provide a toe realignment and toe protecting device which includes a dorsal and plantar piece and connecting arms between the toes where the dorsal piece is cylindrical in shape and rests at the bases of the central three toes.

Yet another object of the invention is to provide a toe realignment and toe protecting device which includes a dorsal and plantar piece and connecting arms between the toes where the dorsal piece is cylindrical in shape and rests at the bases of two of the central three toes.

Yet still another object of the invention is to provide a toe realignment and toe protecting device which includes a dorsal and plantar piece and connecting arms between the toes further comprising a buttress modification to straighten hallux valgus or bunion deformities.

The objects of the invention are achieved by provision of a device for the realignment of hammer toe deformities comprising a dorsal piece and a plantar piece parallel thereto. The dorsal piece and the plantar piece are held together by a first and a second connecting arm. The device being of a resilient material and of a unitary construction is suitably sized and shaped to fit around the wearer's foot, within the wearer's shoe in a position wherein the dorsal piece rests across the bases of at least two of the central three toes of the wearer's foot and the plantar piece is tapered laterally and is comma shaped to fit in the sulci formed by the central three toes of the wearer's foot. The first connecting arm extends through the interspace between the hallux and the second toe and the second connecting arm extends through the interspace between the third toe and the fourth toe.

### DESCRIPTION OF THE DRAWINGS

The present invention and the manner in which it may be practiced is further illustrated with reference to the accompanying drawings wherein:

FIG. 1 is a perspective view of a first embodiment of a device to alleviate hammertoe deformities of the present disclosure shown in use.

FIG. 2 is a perspective view a first embodiment of a device to alleviate hammertoe deformities of the present disclosure.

FIG. 3 is a front view of a first embodiment of a device to alleviate hammertoe deformities of the present disclosure.

FIG. 4 is a bottom view of a first embodiment of a device to alleviate hammertoe deformities of the present disclosure shown in use.

FIG. 5 is a top view of a first embodiment of a device to alleviate hammertoe deformities of the present disclosure shown in use.

FIG. 6 is a front section detail view of a first embodiment of a device to alleviate hammertoe deformities of the present disclosure shown in use.

FIG. 7 is a perspective view of a second embodiment of a device to alleviate hammertoe deformities of the present disclosure.

FIG. 8 is a front view of second embodiment of a device to alleviate hammertoe deformities of the present disclosure shown in use.

FIG. 9 is a top view of second embodiment of a device to alleviate hammertoe deformities of the present disclosure shown in use.

FIG. 10 is a front section detail view of second embodiment of a device to alleviate hammertoe deformities of the present disclosure shown in use.

FIG. 11 is a top view of a third embodiment of a device to alleviate hammertoe deformities of the present disclosure.

FIG. 12 is a front section detail view of a third embodiment of a device to alleviate hammertoe deformities of the present disclosure shown in use.

FIG. 13 is a front view of a third embodiment of a device to alleviate hammertoe deformities of the present disclosure.

### DETAILED DESCRIPTION OF THE INVENTION

At the outset, it should be clearly understood that like reference numerals are intended to identify the same structural elements, portions, or surfaces consistently throughout the several drawing Figures, as may be further described or explained by the entire written specification of which this detailed description is an integral part. The drawings are intended to be read together with the specification and are to be construed as a portion of the entire “written description” of this invention as required by 35 U.S.C. § 112.

Hammertoes are a contracture of the toes such that the proximal aspect of the toe sticks up and the distal aspects of the toe points down and curls. These deformities start out flexible and are reduced when the foot is loaded. Over time they become rigid leading to corn formation and, in some cases, ulceration. In many instances the deforming force contributing to formation of hammertoes is a hallux valgus or bunion deformity. The valgus position of the hallux forces the toes to pop up and form hammertoes, various devices have been used to relieve symptoms including crest pads, toe pads, toe caps, toe separators and bunion splints.

The present disclosure provides a digital splinting and toe protecting device that is designed to be worn in the shoe. The device of the present disclosure eliminates pressure on the tops and bottoms of the toes. Further, when worn in shoes, the device applies forces to the top and bottom of the toes which straightens the toes and minimizes, or even eliminates, any pressure on the dorsal joints or the tips of the toes. Straightening the toes prevents the hammertoes from becoming rigid thereby preventing formation of corns or ulcerations. In patients with existing corns or ulcerations due to rigid hammertoe deformities the device eliminates the pressure on those areas allowing the toes to heal. In an alternative embodiment, a buttress modification 132 of the device further applies a force to the hallux, or great toe, to help straighten a bunion deformity.

The device of the present disclosure is designed to be worn in a shoe. The upper and sole of the shoe applies pressure to the dorsal and plantar pieces of the device thereby providing toe realignment in flexible hammertoe deformities. In rigid hammertoe deformities the device keeps pressure off the affected joints preventing corns and ulcers from forming.

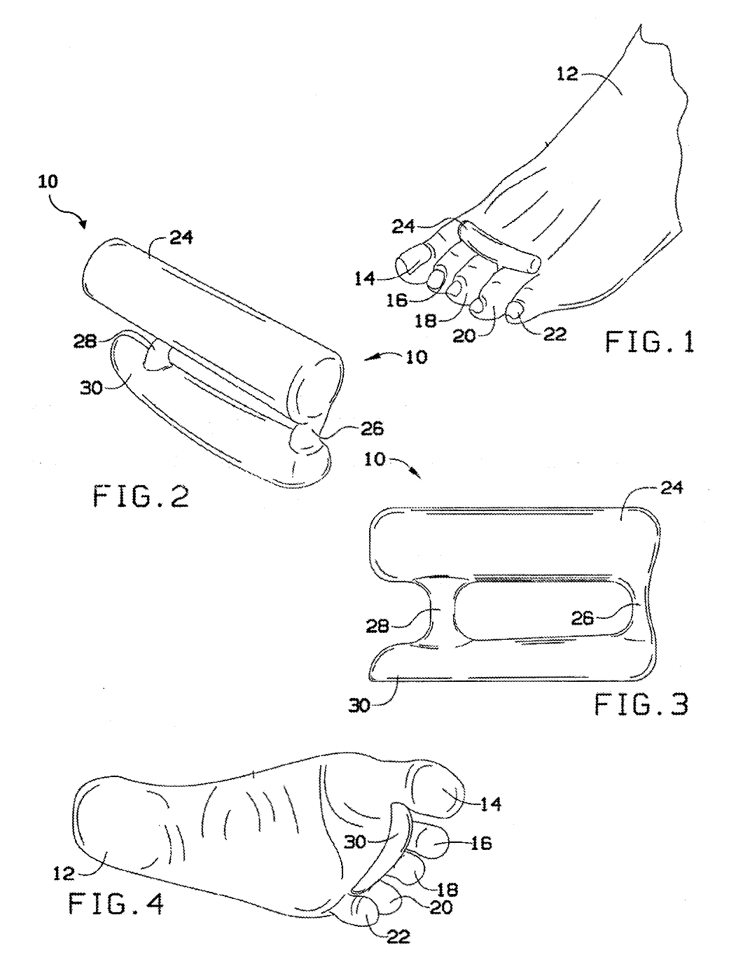

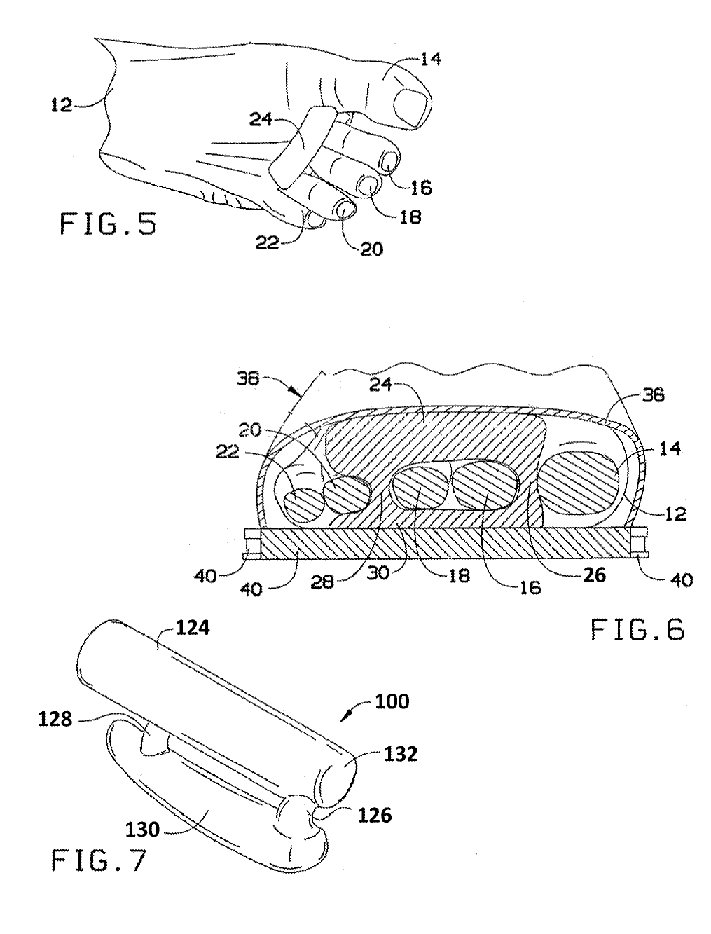

Adverting now to the drawings, with reference to FIGS. 1 through 6, a first embodiment of a device to alleviate hammertoe deformities of the present invention is indicated generally by numeral 10. In the first embodiment, device 10 includes dorsal bar 24 and plantar platform 30. Dorsal bar 24 and plantar platform 30 are connected by hallux arm 26 and toe arm 28. Preferably, dorsal bar 24 is cylindrical in shape and rests at the bases of the central three toes 16, 18 and 20. Dorsal bar 24 is two to three inches in width and one half to one inch in diameter. Plantar platform 30 is tapered laterally and comma shaped so as to fit securely and comfortably in the toe sulci formed by toes 16, 18 and 20. When worn on foot 12 (as seen in FIG. 6), hallux arm 26 is situated between hallux 14 and second toe 16 while toe arm 28 is situated within the third interspace (i.e. between third toe 18 and fourth toe 20). Preferably hallux arm 26 and toe arm 28 are one half to three quarter inches in height and one eighth to one quarter inch in diameter.

Device 10 is constructed of any suitable material, but preferably is constructed as a solitary unit made of silicone, viscoelastic gel or moldable rubber. Device 10 can be created by traditional form-molding techniques. For instance, a manufacturer creates a two-piece, three-dimensional mold of the device. The selected material (silicone, viscoelastic gel, moldable rubber) is heated until it becomes liquid. The liquid is then poured into mold and allowed to cool to create the final product. It is further envisioned that the device can be made in three pieces where moldable rubber or foam encased in a cloth, leather or suede cover is used for the dorsal bar and plantar platform and the arms of the device are made of elastic. The three piece construction would lower the cost of the device but the device would not be as well-secured on the foot.

As can be seen in FIG. 1, dorsal bar 24 sits over the bases of the central three toes 16, 18, and 20. As seen in FIG. 5, the hallux arm 26 and toe arm 28 of the device extend down in the first and third interspaces of the toes, respectively, to accommodate the second and third toes, 16 and 18 respectively, and to help secure the device to the toes. The arms are connected to the comma shaped plantar platform, and as shown in FIG. 4, plantar platform 30 follows the toe sulci and helps to secure the device to the foot.

In the embodiment presented in FIGS. 1 through 6, dorsal bar 24 and plantar platform 30 in conjunction the two arms 26 and 28 are necessary to alleviate hammertoe conditions on the central three toes 16, 18, and 20. The arms of the device can be quickly and easily slipped over the second and third toes. As shown in FIG. 6, when worn in shoe 38, the device straightens the toes and eliminates pressure on the tops and tips of the toes thereby providing relief for corns, calluses or ulcerations. Dorsal bar 24 is fashioned to fit at the bases of the toes to provide a plantar flexor force on the metatarsophalangeal joints. The cylindrical shape of dorsal bar 24, in conjunction with toe box 36 of shoe 38, provides a better plantar flexory force to the metatarsophalangeal joints of central toes 16, 18, and 20 than a flat design. The plantar flexory force keeps the extensor tendons and the dorsal capsular ligaments stretched to prevent rigid contracture. The comma or crescent shaped plantar platform 30 fits securely in the sulci of the toes and prevents movement of the device when walking. Sole 40 exerts an upward force on plantar platform 30 which in turn creates a dorsiflexory force on the middle joint of the toes. This dorsiflexory force straightens the toe and takes pressure off of the tip of the toe. Plantar platform 30 is tapered and fits the contour of the sulci and provides an extension force on the distal joints to prevent further curling of the toes.

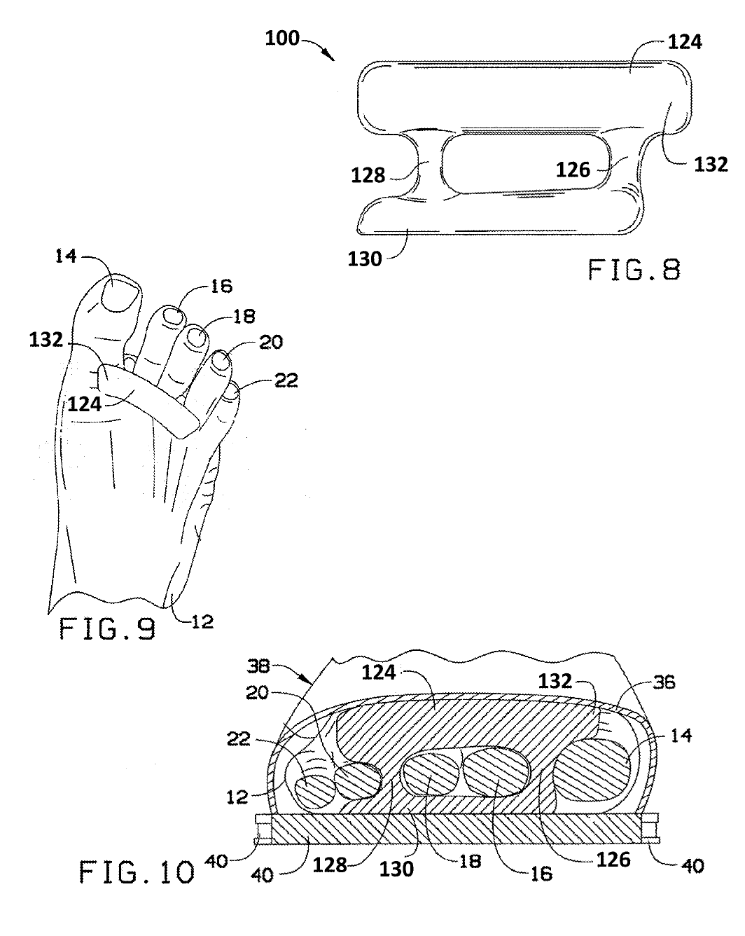

With reference to FIGS. 7 through 10 a second embodiment of a device to alleviate hammertoe deformities of the present disclosure is indicated generally by numeral 100. Hammertoe/bunion device 100 is constructed similarly to device 10 discussed above but is further equipped with buttress modification 132 to dorsal bar 124. With buttress modification 132, hallux arm 26, which rests within the first interspace between hallux 14 and second toe 16, is one half to one inch thick and is curved to exert an adductory force on the hallux 14. Buttress modification 132 helps to straighten hallux valgus or bunion deformities while dorsal bar 124, plantar platform 130, and arms 126 and 128 simultaneously alleviate hammertoe conditions.

Device 100 has toe buttress modification 132 above the first and second toes. As shown in FIG. 10, because hallux 14 is abducted, when shoe 38 exerts a plantar flexory force on the cylindrical dorsal bar 124, lesser toes 16, 18, and 20 are plantar flexed. The plantar flexion of the lesser toes creates an adductory force on hallux 14 due to the presence of buttress modification 132. Buttress modification 132 has a dorsal concave design which further adducts hallux 14 when the plantar flexory force is applied. Thus, when worn in the shoe, device 100 alleviates hammertoe conditions of toes 16, 18, and 20 while also correcting hallux valgus of hallux 14.

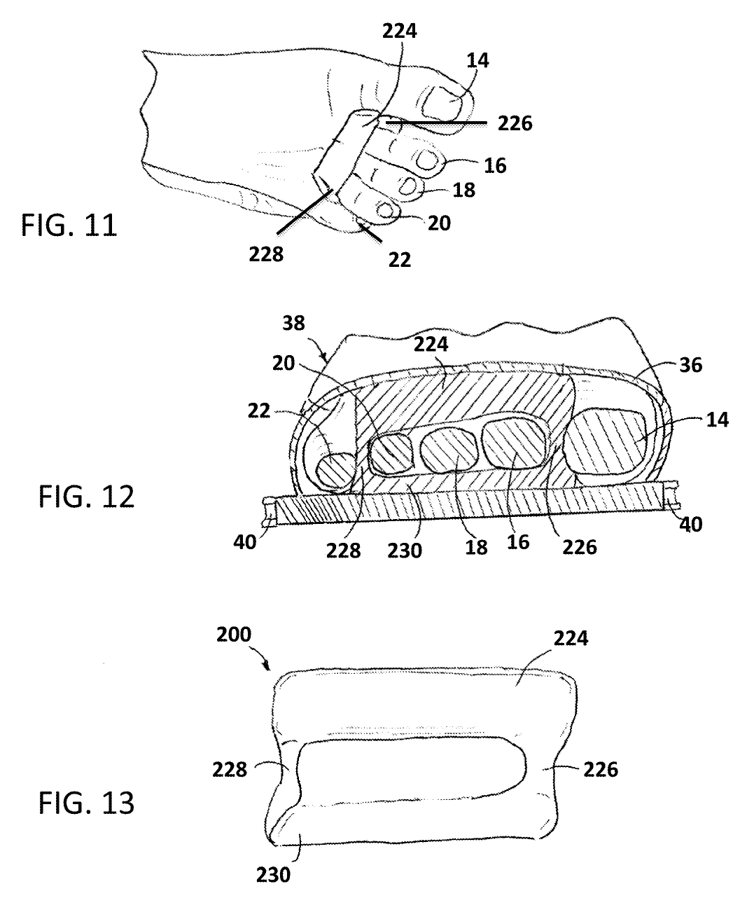

With reference to FIGS. 11 through 13, a third embodiment of the present disclosure is indicated generally by numeral 200 (as seen in FIG. 13). In the third embodiment, device 200 includes dorsal bar 224 and plantar platform 230. Dorsal bar 224 and plantar platform 230 are connected by hallux arm 226 and toe arm 228. Preferably, dorsal bar 224 is cylindrical in shape and rests at the bases of the central three toes. Dorsal bar 224 is two to three inches in width and one half to one inch in diameter. Plantar platform 230 is tapered laterally and comma shaped so as to fit securely and comfortably in the toe sulci. When worn on foot 12, hallux arm 226 is situated between hallux 14 and second toe 16 while toe arm 228 is situated within the fourth interspace (i.e. between fourth toe 20 and pinky toe 22). Preferably hallux arm 226 and toe arm 228 are one half to three quarter inches in height and one eighth to one quarter inch in diameter.

As can be seen in FIG. 11, dorsal bar 224 sits over the bases of the central three toes 16, 18, and 20. As seen in FIG. 12, the hallux arm 226 and toe arm 228 of the device extend down in the first and fourth interspaces of the toes, respectively, to accommodate the second, third and fourth toes, 16, 18 and 20 respectively, and to help secure the device to the toes. The arms are connected to the comma shaped plantar platform 230 and plantar platform 230 follows the toe sulci and helps to secure the device to the foot.

In the embodiment presented in FIGS. 11 through 13, dorsal bar 224 and plantar platform 230 in conjunction the two arms 226 and 228 are necessary to alleviate hammertoe conditions on the central three toes 16, 18, and 20. The arms of the device can be quickly and easily slipped over the second, third and fourth toes. As shown in FIG. 12, when worn in shoe 38, the device straightens the toes and eliminates pressure on the tops and tips of the toes thereby providing relief for corns, calluses or ulcerations. Dorsal bar 224 is fashioned to fit at the bases of the toes to provide a plantar flexor force on the metatarsophalangeal joints. The cylindrical shape of dorsal bar 224, in conjunction with toe box 36 of shoe 38, provides a better plantar flexory force to the metatarsophalangeal joints of central toes 16, 18, and 20 than a flat design. The plantar flexory force keeps the extensor tendons and the dorsal capsular ligaments stretched to prevent rigid contracture. The comma or crescent shaped plantar platform 230 fits securely in the sulci of the toes and prevents movement of the device when walking. Sole 40 exerts an upward force on plantar platform 230 which in turn creates a dorsiflexory force on the middle joint of the toes. This dorsiflexory force straightens the toe and takes pressure off of the tip of the toe. Plantar platform 230 is tapered and fits the contour of the sulci and provides an extension force on the distal joints to prevent further curling of the toes

Although the invention has been described with reference to certain preferred embodiments, it will be appreciated by those skilled in the art that modifications and variations may be made without departing from the spirit and scope of the invention. It should be understood that applicant does not intend to be limited to the particular details described above and illustrated in the accompanying drawings.

What is claimed is:

1. A device for the realignment of hammer toe deformities comprising a dorsal piece and a plantar piece parallel thereto, said dorsal piece and said plantar piece are held together by a first and second connecting arms, said device being of a resilient material and of a unitary construction, suitably sized and shaped to fit around a wearer's foot, within the wearer's shoe in a position wherein said dorsal piece rests across the bases of the central three toes of the wearer's foot and said plantar piece is tapered laterally and is comma shaped to fit in the sulci formed by the central three toes of the wearer's foot, and said first connecting arm extends through the interspace between the hallux and the second toe and said second connecting arm extends through the interspace between the fourth toe and an adjacent toe of the wearer's foot.

2. A device according to claim 1 wherein said resilient material is selected from the group consisting of silicone, viscoelastic gel, and rubber.

3. A device according to claim 2 wherein said dorsal piece is cylindrical in shape.

4. A device according to claim 3 wherein said adjacent toe is the third toe of the wearer's foot.

5. A device according to claim 3 wherein said adjacent toe is the fifth toe of the wearer's foot.

6. A device according to claim 3 wherein the dorsal piece is extended to additionally rest across the base of the hallux.
